(19) United States
(12) Patent Application Publication
(10) Pub. No.: US 2005/0107873 A1
(43) Pub. Date: May 19, 2005

(54) Accommodative intraocular lens and method of implantation

(75) Inventor: Stephen Q. Zhou, Irvine, CA (US)

Correspondence Address: FROST BROWN TODD, LLC, 2200 PNC CENTER, 201 E. FIFTH STREET, CINCINNATI, OH, 45202, US

(73) Assignee: Medennium, Inc.

(21) Appl. No.: 10/990,606

(22) Filed: Nov. 17, 2004

Related U.S. Application Data
(60) Provisional application No. 60/523,504, filed on Nov. 18, 2003.

(51) Int. Cl. A61F 2/16

(52) U.S. Cl. 623/6.13; 623/6.37; 623/6.39

(57) ABSTRACT

An accommodative intraocular lens (IOL) and a method of implanting the lens are disclosed. The lens is made from a soft shape memory material and has a first configuration associated with a first diopter power. When the lens is implanted into the capsule in the eye, the interaction between the lens and the capsule, based on their relative sizes, causes the lens to take on a second configuration with an associated second diopter power. The force placed on the capsule by tensioning and untensioning of the zonules causes the lens to move between its first and second configurations and diopter strengths, thereby providing lens accommodation to the patient.

### CROSS REFERENCE TO RELATED APPLICATION

This application is based up and claims priority from U.S. Provisional Patent Application No. 60/523,504, filed Nov. 18, 2003, incorporated herein by reference.

### TECHNICAL FIELD

The present invention relates to an accommodative intraocular lens (IOL) and its method of implantation into the eye. Specifically, it relates to an IOL which is suitable for implanting into the capsule of an eye through a small incision to replace the natural crystalline lens after its removal and to restore accommodation in the eye. It also relates to a method for implantation of an IOL such that the IOL or at least its optic body is restricted inside the capsule. As a result, the restriction of the IOL causes a change in the shape of the IOL or at least its optic body, which in turn causes a change in the diopter power of the IOL. This change in IOL shape and its diopter power by various degrees of restrictive conditions provide the eye of a patient with improved far vision and/or near vision. Thus, it restores the accommodation of an aged human eye.

### BACKGROUND OF THE INVENTION

A healthy young human eye can focus an object in far or near distance, as required. In order for the lens to focus on an object at a distance, zonules exert their force to stretch the natural crystalline lens so it becomes gradually thinner until the lens focuses on the target object. This state of the eye, with its focus on a distant object is frequently called the unaccommodative state. On the other hand, when near distance vision such as reading a newspaper is required, zonules relax to release their pulling force such that the natural crystalline lens becomes increasingly thick until it focuses on the target object in the near distance. This state of the eye is called the accommodative state. The capability of the eye changing back and forth from near vision to far vision is called accommodation. As we age, a young healthy eye gradually loses its capacity for accommodation. Around the age of 40, the gradual aging of the human natural crystalline lens increases its rigidity to a level where people start to feel the gradual loss of accommodation. By 50, near vision becomes so difficult that reading glasses usually are required. This naturally occurring process of aging, whereby the natural crystalline lens loses its elasticity, thus resulting in the gradual loss of near vision, is called presbyopia.

Since presbyopia happens to most people around the age of 50, there have been tremendous efforts in scientific and industrial fields to find a solution for restoring accommodation. A simple and direct approach is to replace the aged rigid lens with a soft, malleable lens. In order to do this, ophthalmologists have to preserve the integrity of the capsular bag as much as possible. As shown in FIG. 1, the natural crystalline lens 1 is positioned inside the capsule or capsular bag 2 to which zonules 3 are attached in its equatorial perimeter area. There have been attempts to fill the whole capsule with a viscous liquid by injection via a syringe after the natural lens is removed surgically. The viscous liquid then polymerizes in situ within the capsule to form a gel-like material to act as a lens with its shape defined by the capsule. The problem with this method is that the surgeon has no way of controlling a desirable level of refilling to achieve a target optic diopter with sufficient optic resolution. Because in this case the capsule is used as the lens mold to hold the viscous liquid inside, a surgeon has no way to know when to stop refilling the capsule with the viscous liquid. For example, Huo et al. in U.S. Pat. Nos. 6,361,561 and 6,030,416 discloses an injectable silicone with a specific gravity greater than 1. Once the silicone is injected into the capsule where the natural crystalline lens has been previously removed surgically, it cures in situ to form a lens inside the capsule. Because the cured silicone is a soft gel IOL, its focal length can be adjusted according to whether or not the eye is in the accommodative or unaccommodative state. However, it is not known from the disclosure how the injectable silicone can form an IOL inside the capsule such that the IOL formed in situ can provide the precise diopter power for a specific need of an individual patient.

Alternatively, Wang et al. in U.S. Pat. No. 5,316,704 discloses a process for deforming a full size hydrogel IOL into a rod shape which allows for insertion using a small incision. After it is positioned inside the capsule, the rod absorbs water to hydrate into an enlarged elastic form reassuming the original lens configuration of a full capsule size. However, Wang et al. is silent on whether and how his full size expansile IOL can be utilized as an accommodative lens. Separately, Zhou in U.S. Pat. No. 5,702,441 discloses a method for rapid implantation of shape transformable IOLs, including full size IOLs, through a small incision. Nevertheless, Zhou is completely silent on whether or not his method can be used for an accommodative IOL. Lastly, Zhou et al. in PCT publication WO 01/89816 A1 discloses an ophthalmic device, including full size IOLs, made from crystallizable elastomers and a method of implantation for such ophthalmic devices, including accommodative IOLs.

In addition to full size IOL designs, such as those described above, other designs for accommodative IOLs have also been taught in the literature. Numerous U.S. patents, such as U.S. Pat. Nos. 6,391,056; 6,387,126; and 6,217,612 disclose accommodative IOLs with a common design feature, i.e., the effective lens power of a given diopter is dependent on the location of the lens optic body along the optical axis. In other words, if the lens optic body shifts posteriorly along its optic axis, i.e., shifting away from the cornea, the eye can see distance vision, equivalent to the unaccommodative state. If the lens optic body shifts anteriorly, the eye can focus on a near object, equivalent to the accommodative state of the eye. In these cases, the lens diopter power does not change; it is the shifting of its location along the optic axis inside the eye which provides the eye with a new method for achieving near or distance vision. Ultrasound imaging technique has shown that the lens optics can shift along the optic axis within a range of about 1 mm. This approximately relates to an optic power shift of about 1 diopter. Usually, an effective accommodative lens requires a focus power change of 3 diopters, in order to permit a patient to perform near vision tasks, such as reading a newspaper, without difficulties.

Accommodative lens designs with a multiple optic lens assembly have been disclosed in several U.S. Pat. Nos 6,423,094; 5,275,623; and 6,231,603. In these designs, the optic diopter power of the assembly is dependent on the distance between these optic lenses. The optic diopter of an individual lens does not change during the accommodation-unaccommodation process.

There is a need for an IOL which can replace the aged presbyopic crystalline lens with or without cataract and can restore the accommodation of the lens. The accommodative lens of the present invention was designed with its predetermined initial optic diopter targeted at an individual patient's refractive error. Once it is implanted inside the capsule, the accommodative IOL of the present invention is sufficiently soft so that it interacts with and responds to the eye muscle movement in such a way that its optic diopter increases (for near vision) or decreases (for far vision), as needed.

### SUMMARY OF THE INVENTION

The primary object of the present invention is to provide an accommodative IOL for an aged eye with or without cataract. The accommodative IOL is made with predetermined initial optic diopter power and optic resolution. The initial optic diopter of the IOL is targeted for correcting the individual patient's refractive error. The most important feature of the present accommodative IOL design is that it engages with the capsular bag once it is positioned inside the capsule after the aged natural lens is removed. Because the IOL or at least its optic portion is made from a soft material and it has at least one dimension equal to or preferably larger than the corresponding dimension of the capsule, it will change its shape, such as lens curvature or central thickness, according to its engagement force with the capsule. This interaction between the IOL and the capsule allows the IOL to increase or decrease its surface curvature, and thus its diopter power for achieving near vision or far vision, as needed.

Another object of the present invention is to construct the accommodative lens from a biocompatible shape memory material of appropriate softness. The shape memory material will allow the IOL to be implanted through a small incision while the appropriate softness will allow the IOL to change its shape in response to the eye muscle force. Too hard a material will not allow the IOL to change its shape in response to the eye muscle force. Generally speaking, materials suitable for the present application should have softness at least 5 times softer than a typical soft foldable IOL now in the marketplace. This means the proper softness for the accommodative IOL of the present invention has a durometer of no greater than about 5 Shore A, and preferably about 1 Shore A or less.

A further object of the present invention is to provide a method for implanting the accommodative IOL wherein the method comprises (a) providing an accommodative IOL in its first configuration with a predetermined first optic diopter power targeted for the patient's specific refractive errors, and having at least one dimension larger than the corresponding dimension of the patient's capsule; (b) removing the aged natural human crystalline lens from the patient; (c) implanting the IOL inside the capsule wherein the IOL changes from its first configuration to a second configuration due to the restriction of the IOL inside the capsule, resulting in a change in the IOL's optic power from its first dioptic power to a second dioptic power. When the zonules place varying amounts of stress on the capsule during the normal vision process, the lens moves between its first and second diopter strengths. Accordingly, the interchange between the first diopter and the second diopter provides a mechanism for adjusting far vision and near vision. Thus, it restores accommodation for an aged eye.

These objects and others can be achieved as demonstrated by the lenses taught in the following description and preferred exemplary embodiments.

### BRIEF DESCRIPTION OF THE DRAWINGS

FIG. 1 is a representation of the anatomy of a human eye wherein 1 is the natural crystalline lens, 2 is the capsule or capsular bag, 3 are zonules attaching to the capsule in the equatorial region, 4 is the iris, and 5 is the cornea. The natural crystalline lens is an asymmetrical biconvex lens with a typical posterior surface radius of 6-8 mm and an anterior surface radius of 9-12 mm. The dotted line in FIG. 1 is the imaginary axis, or so-called optical axis, passing through the optical center of the eye and perpendicular to the plane of the crystalline lens.

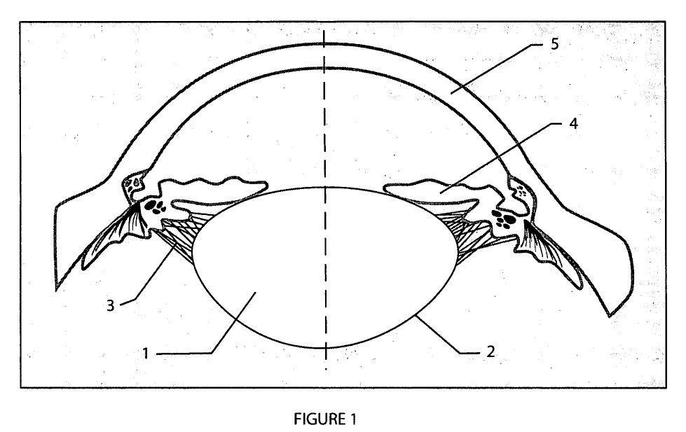

FIG. 2 is an example of an accommodative lens design 6 of the present invention. The diameter of the lens is preferably in the range of from about 8 mm to about 13 mm, even more preferably in the range of about 9.5 to about 11 mm.

FIG. 2A is the full size IOL in its first configuration with a first diopter power.

FIG. 2B is the same IOL positioned inside the human capsule in its accommodative state. FIG. 2B has a second shape with a second optic diopter featuring a larger central lens thickness but smaller lens diameter than that in FIG. 2A.

FIG. 3 is another example of an accommodative lens design wherein the optic body (8) is made of a softer material than the haptic body (9). In this example, the haptic body has a ring-like structure except that a slice of the ring has been cut out (10). This will allow the ring to contract during accommodation thereby forcing the soft central optic body to change into a second configuration with a second diopter. Once the contraction force is relieved during unaccommodation, the central optic body will recover to its initial first configuration with the first diopter power. In order to have a solid contact between the central optic body and the ring-like haptic body, the inner diameter of the haptic body has to be same as or slightly smaller than the out diameter of the central optic body.

FIG. 4 is the side view of the accommodative lens shown in FIG. 3.

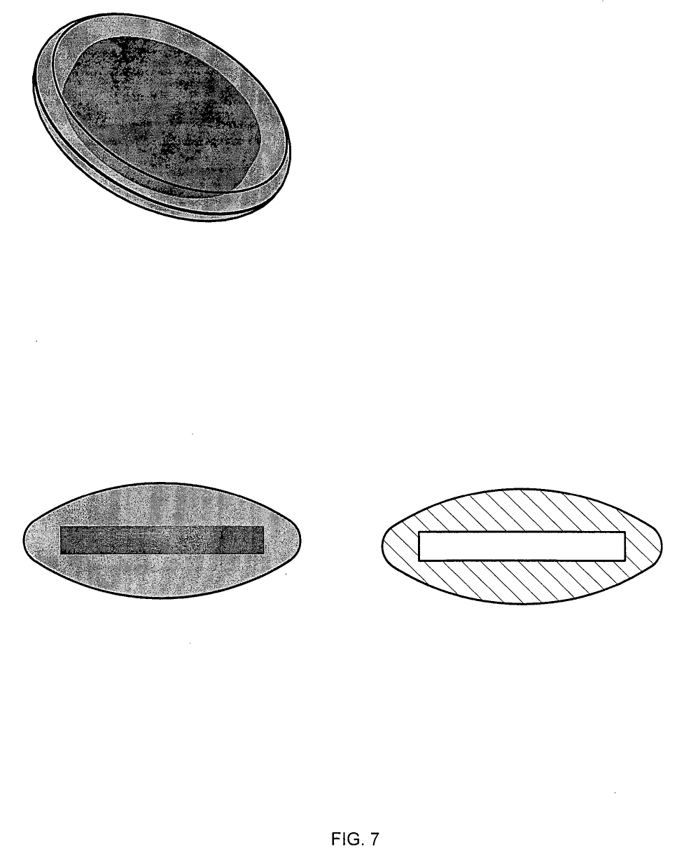

FIG. 5 is still another example of an accommodative full sized lens design wherein the soft core portion (11) of the IOL is surrounded and sealed by the outside skin layer portion (12) made from a shape-memory material. The soft core portion of the IOL is comprised of a fluid, a gel, or in the extreme case, a gas. It is not necessary for the soft core portion to be made from a shape-memory material. The shape of the core portion can be various geometric configurations, which include, but are not limited to biconvex (FIG. 5), biconcave (FIG. 6), convex-concave, plano-plano (FIG. 7), plano-convex, plano-concave or other possible combinations (FIG. 8).

FIG. 9 is another example of an alternative biconvex full size lens design similar to the IOL in FIG. 5 except that it includes a rim surrounding the equatorial periphery area of the IOL.

FIG. 10 is still another example of an alternative design similar to that in FIG. 5 except that the IOL has a flatter anterior surface curvature than the posterior surface.

FIG. 11 is a synthetic human capsule made from a transparent silicone. FIG. 11A is a top perspective view and FIG. 11B is a side view. This device is described in Example 1.

### DETAILED DESCRIPTION OF THE INVENTION

Throughout the disclosure, a “small incision” usually means an incision size in the range of about 3-4 mm for cataract surgery. The first generation of IOLs were made from rigid material, such as poly(methyl methacrylate) with an optic body of approximately 6 mm in diameter. These rigid lenses usually require at least a 6 mm incision in the cornea for implantation into the eye. Since foldable elastic materials were used for the preparation of IOLs, the 6 mm optic body can be folded in half and can be inserted through an incision of about 3-4 mm.

The terms “full size lens” and “full size IOL” are used herein interchangeably. They mean an artificial lens which mimics the natural crystalline lens shape with a lens diameter in the range of about 8-13 mm, preferably in the range of about 9.5-11 mm. The central lens thickness of a full size biconvex (symmetrical or asymmetrical) lens is normally in the range of about 2-5 mm and can be adjusted according to the individual patient's refractive error. A symmetrical biconvex lens means the anterior and posterior surfaces have an identical radius while an asymmetrical biconvex means the anterior surface has a different radius than the posterior surface, such as in the case of a human crystalline lens. Because such a full size lens has a large optical diameter, it usually does not have edge glare, halo or any other optic defects typically associated with a small optic body lens. In addition, a fill size lens can avoid lens decentration, a problem associated with a regular IOL having a 6 mm optic body.

Capsulorhexis is the opening surgically made by puncturing, then grasping and tearing a hole in the anterior capsule. In a regular extracapsular cataract extraction (ECCE) procedure, a capsulorhexis is made in the anterior capsule and the cloudy cataract lens is extracted by phacoemulsification. Obviously, the accommodative IOL of the present invention can be used for patients after cataract surgery. It can also be used for patients with only presbyopia, but without cataract.

The term diopter (D) is defined as the reciprocal of the focal length of a lens in meters. For example, a 10 D lens brings parallel rays of light to a focus at 1/10 meter. After a patient's natural crystalline lens has been surgically removed, surgeons usually follow a formula, based on their own personal preference, to calculate a desirable diopter power (D) for the selection of an IOL for the patient to correct the patient's preoperational refractive error. For example, a myopia patient with −10 D undergoes cataract surgery and IOL implantation; the patient can see at a distance well enough even without glasses. This is because the surgeon has taken the patient's −10 D near-sightedness into account when choosing an IOL for the patient.

The term “dimension of a patient's crystalline lens” is used herein interchangeably with the term “dimension of a patient's capsule.” The dimension of a patient's crystalline lens in the accommodative state or unaccommodative state can be measured using well-known modern techniques.

Shape memory materials are stimuli-responsive materials. They have the capability of changing their shape into a temporary shape under an external stimulus. The stimulus can be, for example, a temperature change or the exerting of an external compression (or stretching) force. Once the external stimulus is eliminated, the shape memory material will change back into its initial shape. A recent review paper of “Shape-Memory Polymers” was published in Angewandete Chemie, International Edition 41(12) 1973-2208 (2002), and is herein incorporated by reference.

The accommodative IOL of the present invention, in one of the preferred embodiments, is made from a shape-memory material and has a sufficient optic resolution and a predetermined optic diopter power tailored for a specific patient's refractive error. The accommodative IOL has its initial first configuration with its first diopter (D₁). The most important feature of the present accommodative IOL design is that the IOL in its first configuration engages with the capsule once it is implanted inside the capsule after the aged natural lens is removed. Because the IOL or at least its optic portion is made from a shape-memory material with an appropriate softness, the interaction of the IOL with the capsule will force it to change into a second configuration having a second diopter (D₂). The degree in the lens shape change as well as the diopter change is dependent on its softness and its engagement force with the capsule.

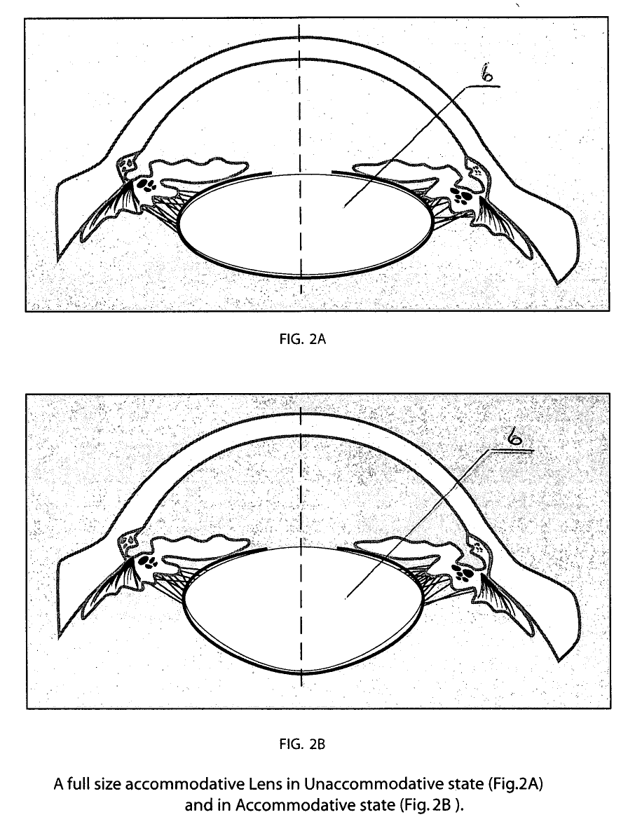

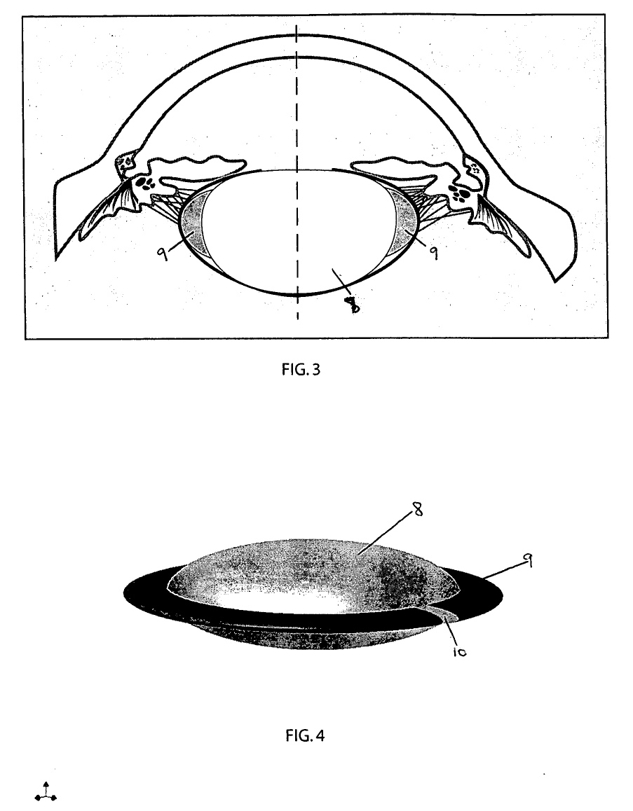

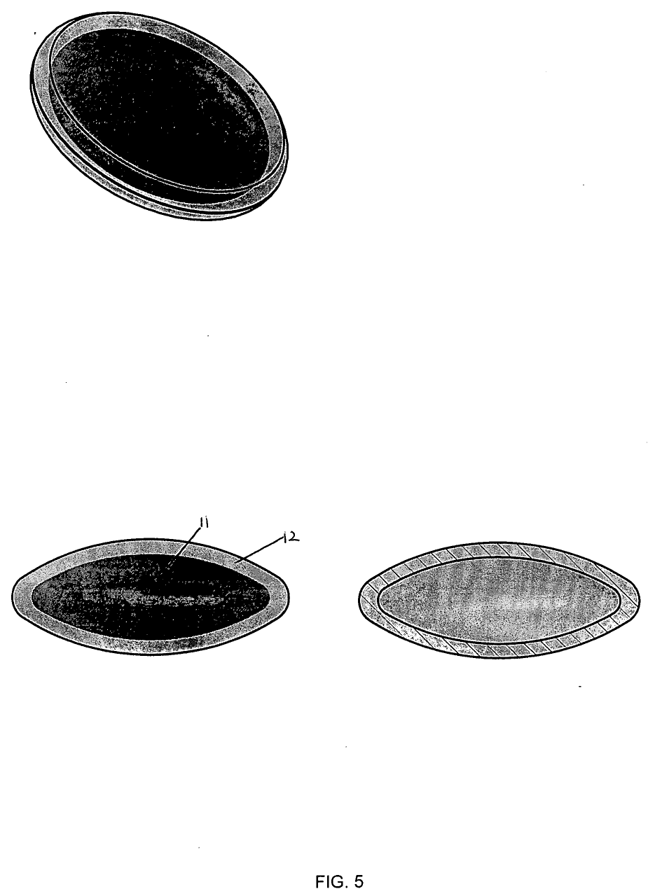

In order to demonstrate the teaching of the present invention, an example is given as follows. A full size accommodative IOL, such as the one in FIG. 2A, has a first configuration with a first diopter power tailored for an individual patient's refractive error for far vision, assuming a 20 D IOL is desirable for a specific patient's far vision. Once the IOL is implanted inside the capsule with the compression force by the capsule, the IOL in FIG. 2A will change into a second configuration with a second optic diopter. In this particular case, the second configuration of the IOL has a smaller diameter but thicker central lens thickness. Therefore, this second configuration and its corresponding second diopter has been increased, for example, to 23 D, as it is shown in FIG. 2B. In this particular case, the IOL configuration in FIG. 2A is for the patient's far vision and the FIG. 2B configuration for the near vision. In a typical situation, a difference of 3 diopters between the IOL's first configuration and second configuration is sufficient for providing for both near and far vision needs of a presbyopia patient. It is known that a human crystalline lens can change its diameter by up to about 1 mm depending on whether or not it is in the accommodative state. For the IOL illustrated in FIG. 2, its diameter has to be at least equal to and preferably larger than the capsule diameter of a patient's eye in the accommodative state. Therefore, the IOL of the present invention in its initial first configuration has a diameter which is up to about 1 mm larger than the diameter of the capsule of a patient's eye in its accommodative state.

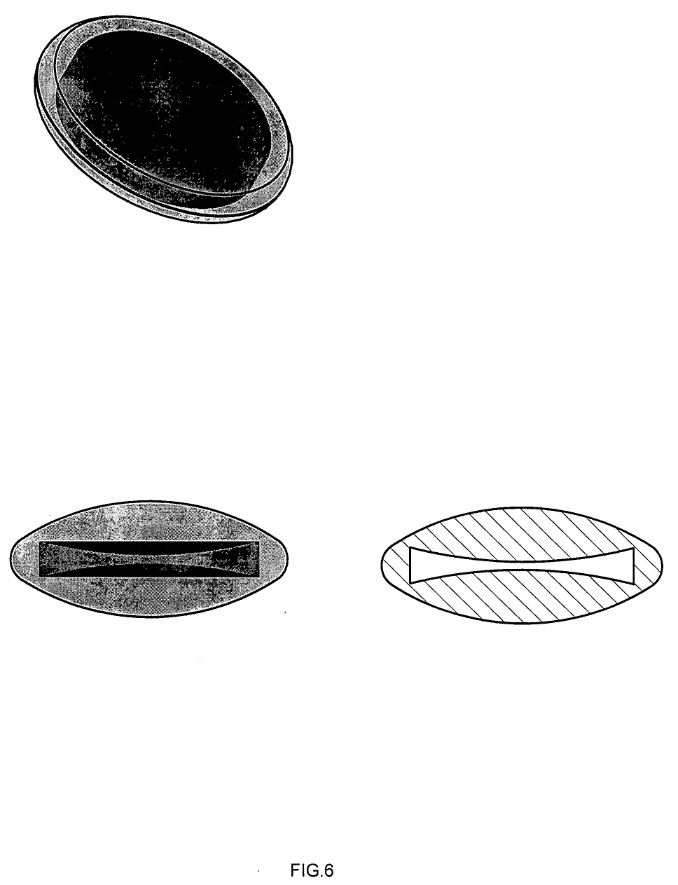

While the above example is only intended for illustrating the teachings of the present invention, it is possible that modified or alternative IOL designs can also be used for achieving the accommodation. For example, the alternative two-part lens design shown in FIG. 3 can be utilized for the same accommodation purpose. In this example, the first part is the central optic body (8) surrounded by the second part haptic body (9) with a ring-like structure except that it is not a closed ring because a slice of the ring structure has been cut out (10). The central optic body is made from a shape-memory material which is soft and susceptible to the compression force, while the ring structure is made from a less soft material. When the capsule contracts during accommodation, the central optic body will be forced to change into a second configuration with a second diopter while the ring simply functions as a medium to transmit the contraction force. When the contraction force is gone during unaccommodation, the central optic body recovers back to its initial first configuration with the first diopter power. The important feature of this design is to allow a surgeon to implant the haptic body and the central optic body separately into the capsule. The diameter of the optic body is preferably in the range from a minimum of about 4.5 mm to approximately 9 mm. In a normal implantation procedure, the ring haptic body is inserted first through a capsulorhexis to stabilize the capsule after the aged natural crystalline is removed. The outer diameter of the ring haptic body is preferably in the range of about 9.5 to 11 mm, although, in extreme cases, it may go as low as about 8 mm or as high as about 13 mm.

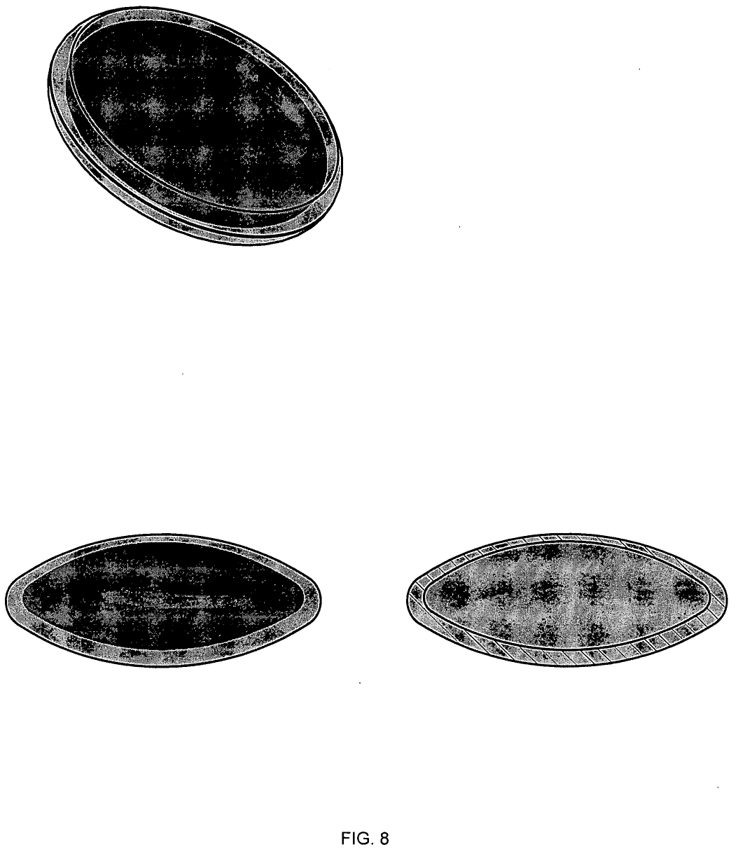

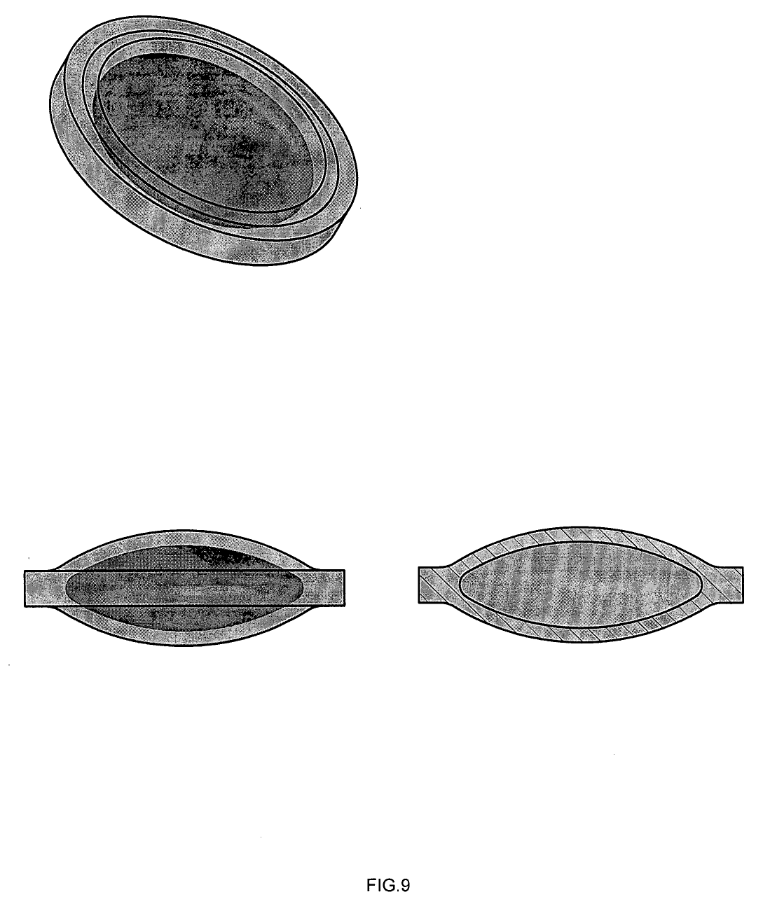

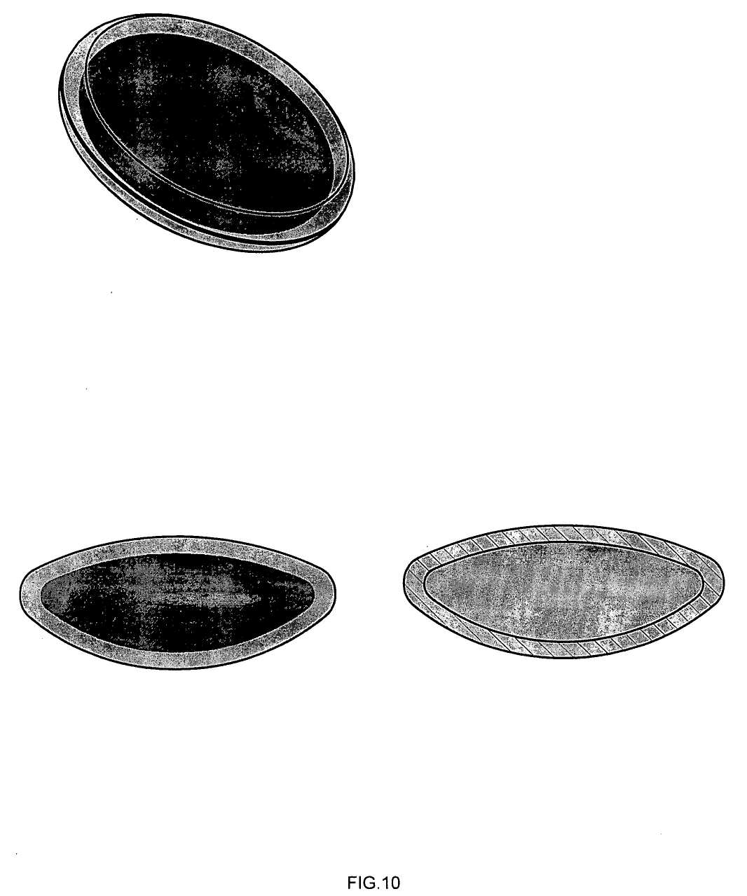

In accordance with another preferred embodiment of the present invention, there is a full size accommodative IOL which can be made from two different materials having different properties, such as softness or refractive index. For example, FIG. 5 is a full size design with the outside skin layer portion (12) made from a shape-memory material and its soft core portion (11) made from a second material with or without shape-memory properties. The shape-memory materials suitable for making the outside skin layer portion include, but are not limited to, acrylic polymers, silicone, collagen containing polymers, and the mixture thereof. Preferably, the skin layer material is selected from shape-memory crystallizable elastomers disclosed in PCT publication WO 01/89816 A1, and herein incorporated by reference. On the other hand, the core portion of the IOL is selected from liquid-like materials comprising (1) inorganic liquids, such as water or saline; (2) organic fluids, such as liquid alkanes, mineral oils, silicone oils, or waxes with melting temperatures at or below about 37° C.; (3) gels, such as silicone gels or hydrogel gels. It is not necessary to have the shape-memory properties for the core portion structure of the IOL. Generally speaking, the thickness of the outside skin layer of the accommodative IOL shown in FIG. 5 can vary from about 0.1 mm to about 2 mm, preferably from about 0.1 mm to about 1 mm, in order to achieve the optimal flexibility such that the eye muscle can effectively change the shape of the IOL once it is implanted inside the capsule. The thickness of the skin layer can be uniform or non-uniform in the posterior surface and in the anterior surface. This composite full size lens design can be used as an accommodative IOL in a similar fashion to the IOL given in FIG. 2, except that the IOL in this case is more susceptible to the eye muscle force because the fluid-like core portion of the IOL has a lower resistance than the IOL made from a homogenous shape-memory material as shown in FIG. 2. The configuration of the core portion of the IOL can be any geometric shape which can provide sufficient optical diopter power for a patient. Possible configurations of the core portion structure include, but are not limited to, biconvex (FIG. 5), biconcave (FIG. 6), convex-concave, plano-plano (FIG. 7), plano-convex, plano-concave or other possible combinations (FIGS. 8-10).

In accordance with another preferred embodiment of the present invention, there is a method for implanting the accommodative IOL into the capsule after the aged crystalline lens is removed. The method comprises (a) providing an accommodative IOL, made from a flexible optical material, in its first configuration having a corresponding first optic diopter (D₁) and resolution predetermined for a patient's specific refractive error, and at least one dimension equal to or (preferably) larger than the corresponding dimension of the patient's capsule; (b) removing an aged human crystalline lens surgically; (c) implanting the accommodative IOL into the patient's capsule wherein the IOL changes from its first configuration to a second configuration due to the restriction of the IOL inside the capsule. This results in a change in the IOL's optic power from its first diopter (D₁) to a second diopter (D₂). The difference between D₁ and D₂ is generally in the range of about 1-5 diopters, preferably in the range about 2-4 diopters, most preferably about 3 diopters.

In order to help in understanding the teachings of the present invention, the following example is given to illustrate the method of implantation for a full size accommodative IOL. It is not intended to limit the scope of the present invention. In step one, a patient's refractive error is measured and the accommodative IOL is decided to have a predetermined diopter power (20 D, for example) for the correction for the patient's refractive error in distance vision. The patient's crystalline lens dimensions are also measured. For example, the diameter of the natural crystalline lens is 9.5 mm in its accommodative state and 10 mm in its unaccommodative state. Accordingly, a full size IOL with a diameter of about 10 mm and with a diopter of 20 D is selected to address the patient's far vision need. In step two, the natural crystalline lens is surgically removed, preferably through a small incision and a small capsulorhexis. In step three, the IOL is implanted into the eye, preferably through a small incision. In this case, the accommodative IOL with a diameter of about 10 mm is forced into a capsule with a 9.5 mm diameter in its accommodative state. Because the IOL is made from a soft material, the compression force by the capsule will cause the IOL to change from its first initial configuration of 10 mm diameter into a second configuration with a reduced diameter but increased lens thickness. This second configuration IOL has a second diopter (D₂=23 D, for example) higher than D (D₁=20 D) in the first configuration. Once the patient's eye focuses on a target in distance (unaccommodative status), zonules stretch the capsule to a larger diameter than that in the accommodative state. Consequently, the IOL will become thinner due to its elasticity and shape-memory properties, and possibly also in part due to the stretching of the IOL by the capsule, providing a lower diopter power (20 D again, for example) for distance vision. It may also be possible that further stretching of the IOL by the capsule leads the lens diopter power to a level smaller than 20 D. Therefore, the IOL of the present invention provides interchangeable diopters, successfully restoring the accommodation for an aged human eye.

For the same hypothetical patient given in the example described in the previous paragraph, it is also feasible to select an accommodative IOL with a diameter of 9.5 mm and with a diopter of 23 D. When such an IOL is selected, the accommodative IOL is referred to as being in the accommodative configuration while the selection of the IOL described in the previous paragraph is referred as being in the unaccommodative configuration. When the IOL selected is in the accommodative configuration, it is dependent on the zonules' stretch to cause the IOL to change from its first configuration with first diopter (D₁=23 D) to the second configuration with the second diopter (D₂=20 D, for example, in this particular case).

The method for the implantation of the present accommodative IOL will ensure the IOL to be engaged with the capsule at all times. When the eye is in the accommodative state, the reduced capsule diameter will force the IOL into its second configuration with a second diopter suitable for the near vision. Once the eye becomes unaccommodative, zonules stretch the capsule to an increased diameter, the accommodative IOL inside the capsule will also increase its diameter mainly due to its elastic property. Accordingly, the IOL becomes thinner and its diopter becomes smaller, suitable for far vision. This accommodation to unaccommodation can be switched back and forth repeatedly, just as in a young accommodative natural eye. It is well known that presbyopia patients still have active zonular stretching movement. It is the natural crystalline lens, which becomes too rigid to change its shape when zonules stretch or relax, which causes the presbyopic condition. The present invention overcomes that problem.

The measurement of the natural crystalline lens dimensions in its accommodative or unaccommodative states can be made with estimation by several literature methods such as the Scheimpflug slit image technique (Dubbelman, Vision Research, 2001; 41:1867-1877), and IR video photography (Wilson, Trans. Am. Ophth. Soc. 1997; 95:261-266), both of which are incorporated herein by reference.

One requisite for the accommodative IOL in the present invention is the selection of a shape memory material with appropriate softness. All the IOLs currently on the marketplace have a durometer hardness of at least 25 Shore A. For example, the best selling lens is Alcon's ACRYSOF® family IOLs with the durometer of 45 Shore A (Source: Product Monograph by Alcon Surgical). Similarly, soft silicone IOLs have a durometer of 38-40 Shore A (Christ et al, U.S. Pat. No. 5,236,970) and a relatively low durometer hardness for silicone IOL material was disclosed to be 28-30 Shore A in U.S. Pat. No. 5,444,106 by Zhou et al. Materials suitable for the present invention should have a hardness in durometer Shore A at least about 5 times softer than those used in the regular IOL applications. This means the durometer hardness desirable for the accommodative IOL will be no greater than about 5 Shore A, preferably about 1 Shore A or less. Suitable materials for the preparation of the accommodative IOLs of the present invention include, but are not limited to, acrylic polymers, silicone elastomers, hydrogels, composite materials, and combinations thereof.

The following examples are intended to be illustrative of, but not limiting of, the present invention.

### EXAMPLE 1

### The Preparation of a Synthetic Human Capsule

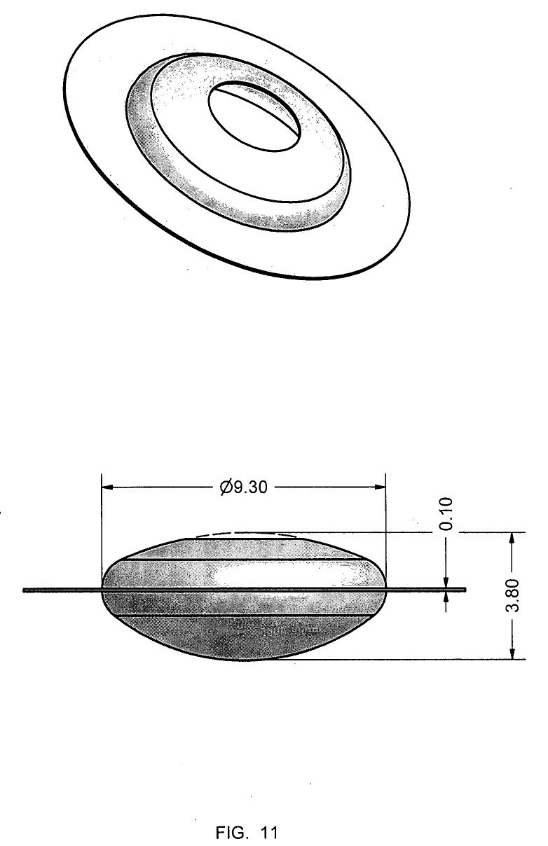

A synthetic human capsule (FIG. 11) is made from NuSil MED 6820 silicone. The capsule has an inner equatorial diameter of 9.3 mm, vertical central thickness of 3.8 mm with posterior radius of 7 mm and anterior surface of 10 mm. Both posterior wall thickness and anterior wall thickness is about 0.1 mm, mimicking the natural human capsule. The capsule also has a 3.8 mm capsulorhexis in the central area of the anterior surface. In addition, the capsule has a thin (about 0.1 mm) flange around the equator that can be clamped in a retaining ring to fix the capsule in position. The capsule is transparent, with 99% visible light transmission.

### EXAMPLE 2

### The Preparation of Accommodative IOLs of Various Dimensions

Into a fused silica mold is added a pre-gel prepared from the mixture of stearyl methacrylate (54% by weight), lauryl acrylate (45% by weight), and 1% of UV absorber, 2-(2′-hydroxy-5′acryloxypropylenephenyl)-2H-benzotriazole, as well as 0.075% of crosslinker, ethylene glycol dimethacrylate. The mold is placed in a pre-heated oven at 110° C. for 16 hours. After the mold is taken out from the oven and cools down to room temperature, the mold is placed in a refrigerator for about 2 hours. The mold is then opened, and a white or translucent solid IOL is carefully removed from the mold. In this way, two different dimensions of accommodative IOLs are prepared. The first group has a diameter of 9.0 mm, central lens thickness of 3.0 mm, and edge thickness of 1.0 mm with an optical diopter power of 27 D, while the second group has a diameter of 9.9 mm, central lens thickness of 2.3 and edge thickness of 1.0 mm with an optical diopter power of 15 D. The durometer hardness of the lenses from both groups is 4 Shore A.

### EXAMPLE 3

### Accommodation Simulation of the First Group Lens

The first group lens has its initial diopter power of 27 D (resolution efficiency of 45.1%) measured with a Meclab Optical Bench using 550 nm wavelength light, 150 mm collimator, 3 mm aperture and 1951 US Air Force Target. The IOL has a central lens thickness of 3.0 mm, lens diameter of 9.0 mm, and edge thickness of 1.0 mm, as measured with a Nikon V12 optical comparator. The same measurement method is used for Example 4. After this lens is implanted into the simulated human capsule described in Example 1, the resolution and diopter power are measured again. It is found that the lens in the capsule has changed its diopter power. The new diopter power in the capsule is 30 D, a shift of 3 D from its initial diopter. The resolution efficiency of the lens inside the capsule is 40.3%. The diopter increase in this case is due to the fact that the lens edge thickness (1.0 mm) is larger than its corresponding dimension of the capsule (about 0.2 mm). This oversized edge thickness forces the soft IOL to move some of its volume toward the central lens area where it has the least resistance due to the presence of the capsulorhexis. Consequently, the central lens thickness has been increased and so has the lens diopter power.

### EXAMPLE 4

### Accommodation Simulation of the Second Group Lens

The second group lens has diopter power of 15 D (resolution efficiency of 51%) with a central lens thickness of 2.3 mm, lens diameter of 9.9 mm, and edge thickness of 1.0 mm. After this lens is implanted into the simulated human capsule described in Example 1, the resolution and diopter power are measured again. It is found that the diopter power of the IOL inside the capsule is 20 D with a resolution efficiency of 40%. The big diopter shift (5 D) in this case is due to the fact that both the lens diameter (9.9 mm) and the lens edge thickness (1.0 mm) are oversized in comparison with the corresponding dimensions of the capsule (9.3 mm and about 0.2 mm respectively). The restriction force by the capsule causes the IOL to change from its first configuration into its second configuration which has a central lens thickness of about 3.0 mm and equatorial diameter of 9.5 mm.

1. An accommodative IOL, made from a shape-memory material, for implantation inside a patient's capsule from which the aged crystalline lens has been surgically removed, wherein said IOL:
(a) has a first configuration with a predetermined first optic diopter defined mainly by the curvatures of the IOL's anterior and posterior surfaces; and
(b) is structurally adapted to change into a second configuration with a second optic diopter due to the interaction of said IOL in said first configuration with said patient's capsule.

2. The accommodative IOL of claim 1 wherein said IOL has a diameter of from about 8 to about 13 mm.

3. The accommodative IOL of claim 1 wherein said IOL has a central lens thickness of from about 2 to about 5 mm.

4. The accommodative IOL of claim 1 wherein said IOL has a diameter at least equal to or larger than the diameter of said patient's capsule in its accommodative state.

5. The accommodative IOL of claim 4 wherein said IOL has a diameter larger than the diameter of said patient's capsule in its accommodative state by up to about 1 mm.

6. The accommodative IOL of claim 1 wherein said IOL has an edge thickness at least equal to or larger than the corresponding dimension of said patient's capsule.

7. The accommodative IOL of claim 1 wherein said first optic diopter is selected for the correction of a patient's far vision and said second optic diopter is for patient's near vision need.

8. The accommodative IOL of claim 1 wherein said shape-memory material is selected from hydrophobic acrylic polymers, hydrogels, silicone elastomers, and combinations thereof.

9. The accommodative IOL of claim 8 wherein said shape-memory material has a durometer of no greater than about 5 Shore A.

10. The accommodative IOL of claim 9 wherein said shape-memory materials has a durometer of no greater than about 1 Shore A.

11. The accommodative IOL of claim 1 wherein said IOL is comprised of (1) the optic body and (2) a haptic body surrounding the equatorial periphery of said optic body, said optic body and said haptic body capable of being implanted through a small incision in said capsule.

12. An accommodative IOL according to claim 1 comprising (1) a soft core portion, and (2) a skin layer portion made from a shape-memory material, wherein said soft core portion is completely surrounded and sealed by said skin layer portion.

13. The accommodative IOL of claim 12 wherein said IOL has a diameter of from about 8 to about 13 mm.

14. The accommodative IOL of claim 12 wherein said IOL has a central lens thickness of from about 2 to about 5 mm.

15. The accommodative IOL of claim 12 wherein said first optic diopter is selected for the correction of a patient's far vision and said second optic diopter is for the correction of a patient's near vision.

16. The accommodative IOL of claim 12 wherein said core portion comprises inorganic liquids selected from water, salines, and mixtures thereof.

17. The accommodative IOL of claim 12 wherein said core portion comprises organic liquids selected from liquid alkanes, oils, waxes with a melting temperature at or below about 37° C., and mixtures thereof.

18. The accommodative IOL of claim 12 wherein said core portion comprises viscous gels selected from silicone gels, hydrogels, and mixtures thereof.

19. The accommodative IOL of claim 12 wherein said skin layer comprises a shape-memory material selected from acrylic polymers, silicones, collagen-containing polymers, and combinations thereof.

20. The accommodative IOL of claim 12 wherein said core portion is thicker in the equatorial area than in the anterior and posterior surfaces.

21. The accommodative IOL of claim 12 wherein said core portion has a thickness of from about 0.1 to about 2 mm.

22. The accommodative IOL of claim 21 wherein said core portion has various thicknesses in the anterior and/or posterior surfaces.

23. A method of implanting an accommodative IOL, made from a shape-memory material, into a patient's capsule from which the aged crystalline lens has been surgically removed, comprising the steps of:
(a) providing said IOL having a first configuration with a predetermined first optic diopter based on the correction of said patient's refractive error, and wherein said first configuration has at least one dimension equal to or larger than the corresponding dimension of said capsule; and
(b) implanting said IOL into the capsule wherein said IOL is forced to change into a second configuration with a second optic power due to the interaction between said IOL and said capsule.

24. The method of claim 23 wherein said IOL has a diameter of from about 8 mm to about 13 mm.

25. The method of claim 23 wherein said IOL has a central lens thickness of from about 2 to about 5 mm.

26. The method of claim 23 wherein said IOL is selected by choosing said first optic diopter based on the correction of said patient's far vision error, and said second configuration with said second optic diopter based on said patient's near vision error.

27. The method of claim 23 wherein said IOL has a diameter at least equal to or larger than the diameter of said patient's capsule in its accommodative state.

28. The method of claim 27 wherein said IOL has a diameter larger than the diameter of said capsule in its accommodative state by up to about 1 mm.

29. The method of claim 23 wherein said IOL has an edge thickness at least equal to or larger than the corresponding dimension of said capsule.

30. The method of claim 23 wherein said shape-memory material is selected from hydrophobic acrylic polymers, hydrogels, silicone elastomers and combinations thereof.

31. The method of claim 30 wherein said shape-memory material has a durometer of no greater than about 5 Shore A.

32. The method of claim 31 wherein said shape-memory material has a durometer of no greater than about 1 Shore A.

33. The method of claim 23 wherein said IOL is comprised of (1) the optic body and (2) a haptic body surrounding the equatorial periphery of said optic body, wherein said optic body and said haptic body are capable of being implanted through a small incision in said capsule.

34. The method of claim 23 wherein said IOL is comprised of (1) a soft core portion; and (2) a skin layer portion made from a shape-memory material, wherein said soft core portion is completely surrounded and sealed by said skin layer portion.

35. The method of claim 34 wherein said core portion comprises inorganic liquids selected from water, salines, and mixtures thereof.

36. The method of claim 34 wherein said core portion comprises organic liquids selected from liquid alkanes, oils, waxes with a melting temperature at or below about 37° C., and mixtures thereof.

37. The method of claim 34 wherein said core portion comprises viscous gels selected from silicone gels, hydrogels, and mixtures thereof.

38. The method of claim 34 wherein said skin layer portion comprises a shape-memory material selected from acrylic polymers, silicones, collagen-containing polymers, and combinations thereof.

39. The method of claim 34 wherein said core portion is thicker in the equatorial area than in the anterior and posterior surfaces.

40. The method of claim 34 wherein said core portion has a thickness of from about 0.1 to about 2 mm.

41. The method of claim 34 wherein said core portion has various thickness in the anterior and/or posterior surfaces.
